(19) United States
(12) Patent Application Publication
(10) Pub. No.: US 2004/0019060 A1
(43) Pub. Date: Jan. 29, 2004

(54) Sigma receptor ligands and their medical uses

(76) Inventors: Barbara Ann Spruce, Dundee (GB); Suzanne Amy Eccles, Sutton (GB); Michael Dexter, London (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/240,327

(22) PCT Filed: Apr. 2, 2001
(86) PCT No.: PCT/GB01/01495

(30) Foreign Application Priority Data
Mar. 31, 2000 (GB) 0007842.8

(51) Int. Cl. A61K 31/496; A61K 31/155

(52) U.S. Cl. 514/253.08; 514/634

(57) ABSTRACT

The present invention is based on the finding that sigma receptor ligands can modulate endothelial cell proliferation and/or survival, and hence control angiogenesis, and in particular that sigma receptor ligand antagonists can be used to inhibit angiogenesis aid so treat conditions such as psoriasis, diabetic retinopathy and cancer. Exemplary compounds include IPAG and rimcazole.

### FIELD OF THE INVENTION

The present invention relates to the use of sigma receptor ligands to modulate endothelial cell proliferation and/or survival, thereby controlling angiogenesis.

### BACKGROUND OF INVENTION

In the normal adult body, most endothelial cells are quiescent, entering mitosis only in response to tissue injury or during menstruation and parturition. However, in pathological states including psoriasis and diabetic retinopathy, endothelial cells may proliferate leading to angiogenesis, the development of new blood vessels. It is also now well recognised that for any cancer to grow beyond a few millimetres in diameter, neoangiogensis is essential and that tumour cells secrete a variety of angiogenic cytokines which stimulate endothelial cell proliferation. One of the most important is vascular endothelial growth factor (VEGF) which also increases the permeability of newly formed vessels. Thus, in cancer, angiogenesis is critical for the development of solid cancers and also provides the conduit through which tumour cells may spread to other parts of the body. However, since a small area of capillary can provide nutrients for a relatively large volume of surrounding cancer cells, any inhibition of endothelial cell proliferation will “amplify” the effect on tumour cells, making this a promising approach for the treatment of cancer. Several different anti-angiogenic agents have been shown to be potent inhibitors of tumour growth and spread.

WO00/00599 discloses that opioid-like agents, including sigma receptor ligands, can be used to cause preferential cell cycle division arrest and apoptosis in populations of diseased cells as compared to normal cells, and in particular that apoptotic effects tend to be greater in tumour cells as compared to normal, non-diseased cells. These effects were demonstrated in this application in in vitro experiments using pure cultures of tumour cells. The results show that normal cells are insensitive to induction of cell cycle division arrest and apoptosis at doses of sigma receptor ligands that are lethal or cytostatic to tumour cells.

WO96/06863 discloses that abrogation of opioid-mediated survival induces apoptosis in cells which are “self-reliant” that is to say they are able to survive by provision of self-generated (autocrine) factors. These cell types are uncommon in normal tissues; an example of a self-reliant cell is the lens epithelial cell type. WO96/06863 and WO00/00599 disclose that “self-reliant” normal cells and tumour cells are unduly reliant on survival mediated through opioid-like agents. Other normal cell types were predicted to retain “back-up” survival mechanisms due to selective pressure to maintain these; this would render them less sensitive to abrogation of opioid and/or sigma mediated survival. Examples were provided in both applications to illustrate the reduced responsiveness of normal cells to abrogation of opioid and/or sigma-mediated survival.

It remains a problem in the art to find substances which are capable of selectively inhibiting endothelial cell proliferation and/or survival.

### SUMMARY OF THE INVENTION

Broadly, the present invention is based on the finding that sigma receptor ligands can modulate endothelial cell proliferation and/or survival, and hence control angiogenesis, and in particular that sigma receptor ligand antagonists can be used to inhibit angiogenesis and so treat conditions such as psoriasis, diabetic retinopathy and cancer. This is surprising as endothelial cells are normal cells rather than the types of cells described in the prior art, namely self-reliant cells or tumour cells, and so it is unexpected that this cell type is sensitive to inhibition of opioid and/or sigma-mediated survival.

The effect sigma receptor ligands have on endothelial cell proliferation and angiogenesis are not readily predictable from the earlier experiments described in WO00/00599 in which sigma receptor ligands were shown to cause apoptosis or cell cycle division arrest in tumour cell cultures as no endothelial cells were present in these cultures. Accordingly, the present invention provides a new way of treating conditions requiring the inhibition or stimulation of endothelial cell proliferation and/or survival, e.g. in the case of cancer by starving the tumour cells of nutrients and inhibiting metastasis. The ability to target endothelial cells in this way is advantageous as tumour cells are difficult to selectively treat as they are genetically unstable and readily acquire drug resistance. In contrast, endothelial cells are genetically stable and consequently more likely to respond to repeated treatment with drugs such as Rimcazole or IPAG.

Furthermore, the experiments described in the present application were carried out on endothelial cells in the absence of tumour cells; thus, the explanation that an anti-angiogenic effect is an exclusive consequence of a decline in production of pro-angiogenic factors by tumour cells can be discounted. Endothelial cells of the type used in these experiments are non-tumorous; they are also not self-reliant. In WO96/06863 it is explained that most normal cells are restrained within their immediate microenvironment due to their requirement for provision of a specific pattern of multiple survival signals to suppress the cell death programme. However, endothelial cells are unusual compared to most normal cell types in being able to survive “ectopically” in a multitude of microenvironments. Thus, an aspect of the present invention is that cells which survive ectopically are also susceptible to abrogation of opioid and/or sigma-mediated survival.

Accordingly, in one aspect, the present invention provides the use of a sigma receptor ligand for the preparation of a medicament for modulating endothelial cell proliferation and/or survival. Thus, the use of sigma receptor ligands provides a way of controlling angiogenesis or neoangiogenesis, acting as an antagonist and inhibiting angiogenesis, or acting as an agonist and promoting angiogenesis.

In WO00/00599, it is disclosed that alternative sigma receptor subtypes, variants or alternate binding pockets on the same receptor macromolecule may be pro-apoptotic; this was exemplified with a sigma antagonistic ligand. A further aspect of the invention, therefore is that alternative sigma agonists may be anti-angiogenic and alternate sigma antagonists may be pro-angiogenic.

In a further embodiment, the present invention provides a method of modulating endothelial cell proliferation and/or survival, the method comprising administering the sigma receptor ligand to a patient in need of treatment in an amount effective for providing an effective amount of modulation.

Preferably, the sigma receptor ligand is a sigma receptor antagonist which has the property of inhibiting endothelial cell proliferation or angiogenesis.

Preferred examples of sigma receptor ligand antagonists are the compounds Rimcazole and IPAG. Preferred embodiments employ Rimcazole, IPAG or their derivatives, prodrugs or pharmaceutically active salts.

Without wishing to bound theory, the inventors believe that while many cells express sigma receptors, the work described herein shows that unusually among normal rather than diseased cells, endothelial cells, and especially neovascular endothelial cells, are unduly reliant (compared to normal cells) on sigma-mediated survival since they undergo apoptosis in response to similar or even lower concentrations of sigma receptor ligand antagonists than tumour cells.

In a further aspect, the present invention provides a method of identifying a sigma receptor ligand which is an antagonist or agonist capable of modulating endothelial cell proliferation and/or survival, the method comprising:

(a) contacting a test compound with endothelial cells;

(b) determining whether the test compound modulates endothelial cell proliferation and/or survival; and

(c) where a compound inhibits survival and/or proliferation, determining that the test compound does not, or to a substantially lesser extent, inhibit survival and/or proliferation in “normal” cells, i.e. cells with typical properties of survival and proliferation regulation.

In the present invention, the endothelial cells may include large (macro) and small (micro) blood vascular endothelial cells, lymphatic endothelial cells, or populations of cells including one or more of these cell types.

Embodiments of the invention will now be described by way of example and not limitation with reference to the accompanying drawings.

### BRIEF DESCRIPTION OF THE FIGURES

FIG. 1a shows RT-PCR of sigma receptor mRNA in human umbilical vein endothelial cells. FIG. 1b shows the sequence of sigma 1 receptor cloned from human vascular endothelial cells.

FIG. 2a shows the results of a proliferation assay in which human microvascular endothelial cells (HMEC-1) were treated with rimcazole on days 1, 4 and 6, with the assay carried out on day 7. FIG. 2b shows the results of a proliferation assay in which human vascular endothelial cells (HUVEC) were seeded at 5×10³/well, with rimcazole added on days 1, 4 and 6 and an alkaline phosphatase assay carried out on day 7.

FIG. 3 shows that sigma 1 receptor agonists rescue endothelial cells from the effects of an antagonist.

FIG. 4 shows the results of an in vitro angiogenesis assay showing the effect of adding rimcazole.

FIGS. 5a and 5b show angiogenesis assays which quantitate the effect of rimcazole and IPAG on in vitro angiogenesis. FIG. 5c shows the rescue of in vitro angiogenesis inhibition by rimcazole by co-incubation with (+)pentazocine. FIGS. 5d and 5e show that pentazocine reverses the anti-angiogenic effect of rimcazole.

FIG. 6a shows mass spectra which identify the metabolites of rimacazole showing the position of hydroxylation and glucuronidation. FIG. 6b shows the excretion profiles of rimcazole and the major metabolites (glucuronide of hydroxylated rimcazole) in plasma, liver and spleen of mice.

FIG. 7a shows graphs of the effect of rimcazole on sponge angiogenesis in mice:¹³¹IUdR uptake, ⁹⁹TcHMPAO uptake and ³H-DG uptake. FIG. 7b shows the results from a rat “sponge” in vivo angiogenesis model showing that rimcazole and IPAG reduce vascular volume in implanted sponges but not in normal tissues.

FIG. 8a shows that rimcazole inhibits growth of MDA MB 435 breast carcinoma xenografts in a dose dependent manner. FIG. 8b shows that the weight of MDA MB 435 tumours at excision is inhibited by rimcazole. FIG. 8c shows that rimcazole reduces the vascular density in these tumours.

FIG. 9 shows that microvascular endothelial cells are selectively killed by sigma antagonists

FIG. 10 shows that adult dermal fibroblasts display robust resistance over a range of rimcazole concentrations which induce dose-dependent cytotoxicity and cytostasis in microvascular endothelial and tumour cells.

FIG. 11 shows that a prototypic sigma-1 agonist, (+) pentazocine, prevents microvascular endothelial cell death induction by rimcazole and IPAG at equimolar concentrations.

### DETAILED DESCRIPTION

### Sigma Receptor Ligands

Specific sigma receptor ligands bind to sigma receptors in substantial preference to other known receptors such as classical opioid receptors—mu, delta, kappa,—dopamine, serotonin, phencyclidine, and beta—adrenergic receptors. A ligand for a sigma receptor can be identified in accordance with the method disclosed in Vilner et al, Cancer Res., 55(2):408-413, 1995. A wide variety of sigma receptor ligands, including sigma receptor ligand antagonists are known in the art and can be used or tested for use in the present invention.

The binding of a putative sigma ligand to sites on sigma receptors, especially sigma 1 receptors can be measured by comparison to the prototypic sigma ligands such as (+)-pentazocine and 1,3-di-o-tolylguanidine (DTG) (and as described by Walker et al., Pharmacological Reviews, 42:355-400, 1990). Radio or chemically labelled prototype sigma ligands are allowed to bind to sigma receptors in the cell preparation. The amount of labelled prototype sigma ligand displaced by the putative ligand is measured and used to calculate the affinity of the putative ligand for the sigma receptor.

As used herein, “sigma receptor” refers to the different forms of sigma receptors (sigma 1, sigma 2 receptors, etc) and to splice variants thereof. Sigma receptors for use in such assays are disclosed in U.S. Pat, No. 5,863,766 or can be obtained by making a suitable preparation such as a crude membrane portion, using conventional protocols, from a cell type, such as a human tumour cell line, which is known to express sigma receptors. Examples of such cell lines would include; A375 melanoma (Accession No: ECACC 88113005), SK-N-SH neuroblastoma (Accession No: ECACC 86012802) and LNCaP.FGC prostate (Accession No: ECACC 89110211). These cell lines are obtainable from the European Collection of Animal Cell Cultures (Porton Down, England) with reference to the accession numbers shown.

Examples of sigma receptor ligands include:

rimcazole (cis-9-[3,5-dimethyl-1-piperazinyl) propyl]carbazole dihydrochloride).

rimcazole hydrochloride.

IPAG (1-(4-iodophenyl)-3-(2-adamantyl)guanidine.

haloperidol, reduced haloperidol.

BD-1047 (N(−)[2-(3,4-dichlorophenyl)ethyl]-N-methyl-2-(dimethylamino)ethylamine).

BD-1063 (1(−)[2-(3,4-dichlorophenyl)ethyl]-4-methylpiperazine.

1,3-di(2-tolyl) guanidine.

(+)-SKF-10047 ((+)-N-allyl normetazocine).

(+)-pentazocine.

(+)-ethylketocyclazocine.

(+)-benzomorphans such as (+)-pentazocine and (+)-ethylketocyclazocine.

(+)-morphinans such as dextrallorphan.

cis-isomers of U50488 and analogues.

arylcyclohexamines such as PCP.

N,N′-diryl-substituted guanidines such as DTG.

phenylpiperidines such as (+)-3-PPP and OHBQs.

steroids such as progesterone and desoxycorticosterone.

butryophenones.

BD614.

(+/−)-cis-N-methyl-N-[2-(3,4-dichlorophenyl)ethyl]-2-(1-pyrrolodinyl)cyclohexylamine.

antipsychotic and potential antipsychotic drugs, additional to haloperidol and rimcazole, which bind with a moderate to high degree of potency to sigma sites including: perphenazine, fluphenazine, (−)-butaclamol, acetophenazine, trifluoperazine, molindone, pimozide, thioridazine, chlorpromazine and triflupromazine, BMY 14802, BMY 13980, remoxipride, tiospirone, cinuperone (HR 375), WY47384.

antidepressants including amitriptyline and imipramine.

A preferred sigma receptor ligand antagonist is Rimcazole (cis-9-[3,5-dimethyl-1-piperazinyl) propyl] carbazole dihydrochloride), a compound known to have activity as an anti-psychotic, e.g. see U.S. Pat. No. 5,955,459, and as an agent which blocks the activity of cocaine (Menkel et al, Eur. J. Pharmacol., 201:251-252, 1991). A range of Rimcazole variants are known and their structure activity relationship has been investigated (Husbands et al, J. Med. Chem., 42(21):4446-4455, 1999).

Rimcazole can be readily produced by those skilled in the art, e.g. using the following synthesis for Rimcazole dihydrochloride (9-3-((3R, 5S)-3,5 dimethyl-piperazin-1-yl)-propyl-9H-carbazole dihydrobromide).

A synthetic route to produce synthon A can be adapted from Whitmore et al, JACS, 66:725-731, 1944. The synthesis of B can be carried out using the stereospecific synthesis of trans compounds shown in Harfenist et al, JOC, 50:1356-1359, 1985. The two precursors can then be coupled to produce Rimcazole dihydrochloride.

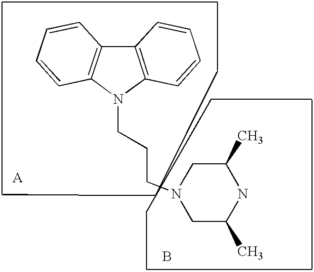

Rimcazole is generally viewed as an antagonistic sigma ligand. For example, Ferris et al (1986 Life Sci Vol 38 pp2329-2337) determined that rimcazole is a specific, competitive antagonist of sigma sites in brain. Rimcazole displays approximately 5-fold selectivity for sigma-1 compared to sigma-2 sites (Abou-Gharbia et al 1993 Annu. Rep. Med. Chem. Vol 28 pp1-10). Thus, rimcazole is classed as a sigma-1-preferring antagonist. The compound IPAG has a high affinity for sigma-1 sites (inhibition constant approximately 2.8nM) and has been described as an antagonist (Whittemore et al 1997 J. Pharm. Exp. Ther. Vol 282 pp326-338).

Activation of sigma-1 receptors antagonises opioid analgesia. Haloperidol was found to potentiate opioid analgesia, an effect that was mediated through its specific sigma binding properties; haloperidol can therefore be regarded as a sigma-1 antagonist (Chien and Pasternak 1995, Neurosci. Lett. Vol 190 ppl37-139). BD-1047 and BD-1063 have a marked selectivity for sigma sites compared to other receptors (opiate, phencyclidine, muscarinic, dopamine, alpha-1-, alpha-2-, beta-adrenoceptor, 5HT-1, 5-HT-2); both drugs bind to sigma-1 and sigma-2 sites but have preferential affinities for sigma-1 compared to sigma-2. Both drugs have been classed as sigma antagonists on the basis of anti-dystonic effects (Matsumoto et al 1995 Eur J Pharmacol Vol 280 pp301-310).

Alteration in the stereochemistry of the kappa agonist trans-U50488 creates the highly specific sigma ligand, cis-U50488 (de Costa et al. 1989 J Med Chem Vol 32 ppl996-2002). Its functional properties in the context of the central nervous system have not been well-defined.

Whereas antagonistic ligands for the sigma receptor may be less well defined, agonistic ligands which have selectivity for the sigma-1 receptor are generally recognised. Prototypic sigma-1 agonists are (+)pentazocine and (+) SKF 10,047 (e.g. Ceci et al 1988 Eur J Pharmacol Vol 154 pp53-57; Maurice and Privat 1998 Neuroscience Vol 83 pp413-428). Sigma-1 agonists are defined as such on the basis of, for example, stimulation of the brain mesolimbic system (Ceci et al) and potentiation of learning and memory (Maurice and Privat).

The inventors reasoned that if rimcazole and IPAG are inducing death at least in part by abrogation of a sigma-1 mediated survival pathway, death should be attentuated by co-administration of the sigma-1 agonists named above. One proviso would be that the sigma-1 agonists would have to access the same subcellular pool(s) of the sigma-1 receptor as rimcazole and IPAG with roughly equal efficiencies. Since the sigma-1 receptor exists on the cell surface as well as at several intracellular sites, the general chemical properties (such as hydrophilicity) of the ligands could affect this result. But it was reasoned that, since sigma-1 agonists named above are effective on neurones, they were likely to be effective as sigma-1 agonists on another “normal” cell type such as microvascular endothelial cells.

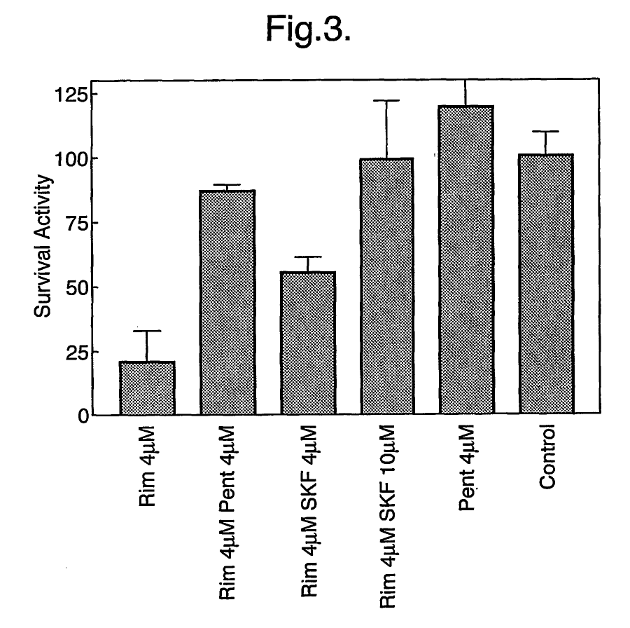

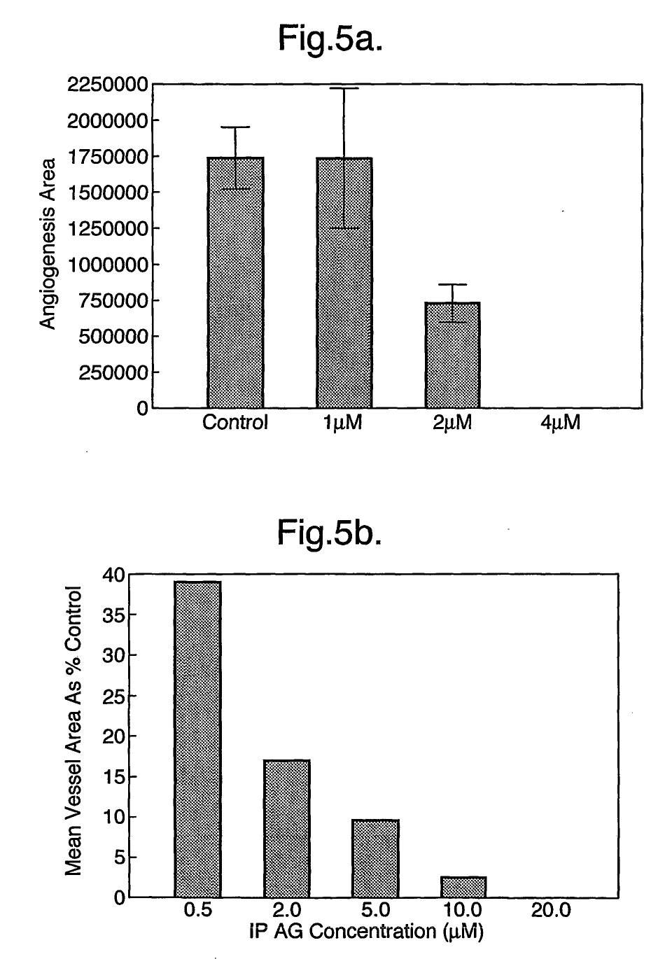

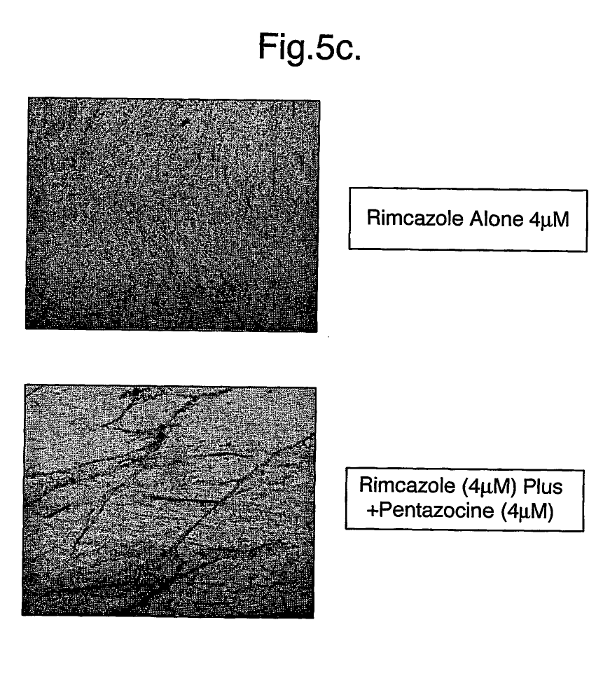

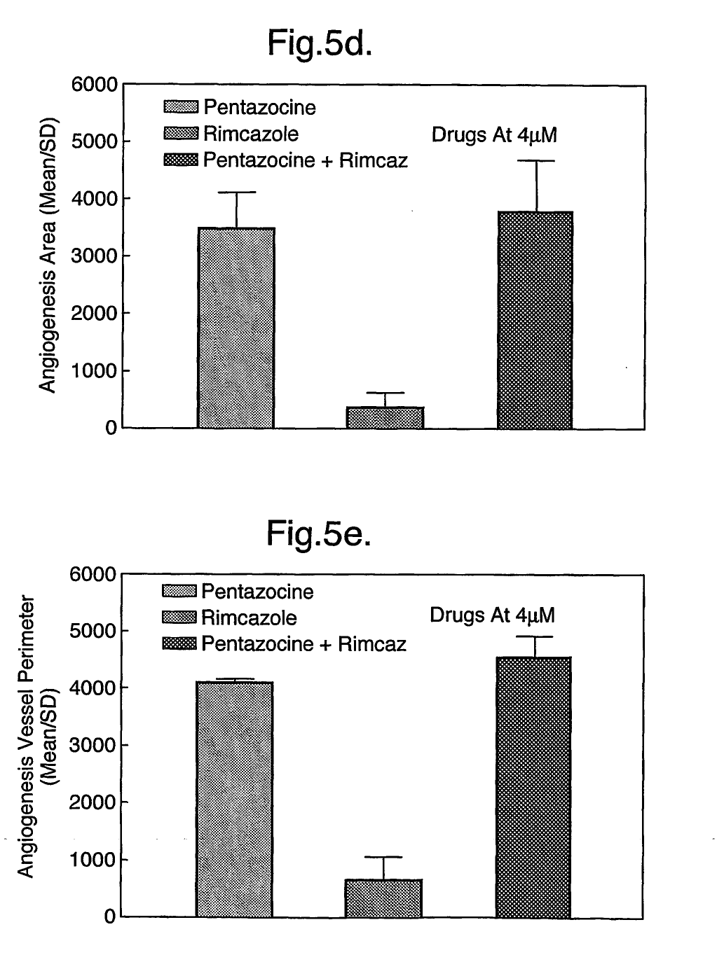

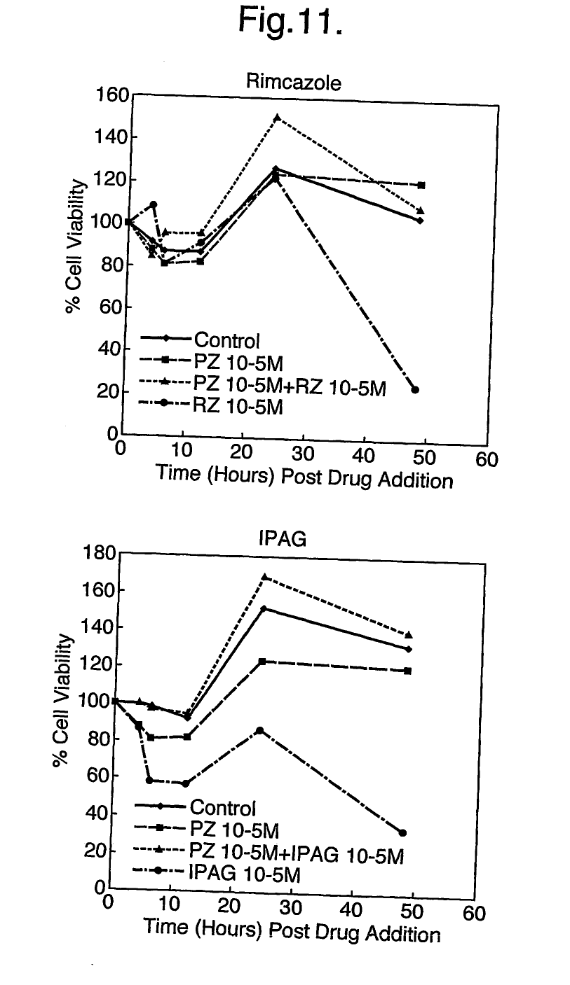

These results described herein show that this is indeed the case. The sigma-1 agonists (+) pentazocine and (+) SKF 10047 prevent or substantially attenuate rimcazole and IPAG-induced death of microvascular endothelial cells and also restore capillary formation; furthermore, rescue was observed with roughly equimolar concentrations of sigma-1 agonists even when the high affinity sigma-1 antagonistic ligand IPAG was used. Exemplification of rescue by sigma-1 agonists was carried out in a number of ways: in different assays of cytotoxicity (FIG. 11); phosphatase-activity (FIG. 3) and also assays of pseudocapillary formation (FIGS. 5c and 5d). These data strongly suggest that the endogenous sigma-1 receptor on microvascular endothelial cells confers an anti-apoptotic drive on which these cells are unduly dependent to restrain the death programme; thus, the programme is unleashed when the sigma-1 pathway is inhibited. It is proposed that the same pathway exists in other normal cells but “back-up” (tissue specific) survival mechanisms prevent the programme from being unleashed in these cells.

The inventors have observed a good correlation between on the one hand the functional properties of sigma ligands which have been defined as agonists or antagonists according to their functions in the central nervous system; and on the other hand, their properties in the context of cell survival. Thus, the prototypic sigma-1 agonists (+)pentazocine and (+) SKF 10047 promote microvascular endothelial cell survival. In contrast, ligands viewed as antagonistic (at least with respect to the sigma-1 receptor) appear to induce death in neovascular endothelial cells, which has been shown to be due to abrogation of a sigma-mediated survival pathway.

It is however well recognised by those skilled in the art of pharmacology in particular that signal transduction events which mediate, on the one hand sigma-mediated functional effects in the nervous system; and on the other hand, cell survival will not necessarily be the same. Thus, until such events have been defined, extrapolation from agonistic and antagonistic action in the context of nervous system functions to action in cell survival must be regarded as indicative but not definitive.

In order therefore to definitively determine whether so-called sigma-1 agonists are indeed promoting an anti-apoptotic function mediated through the sigma-1 receptor, and so-called sigma-1 antagonists are antagonising this anti-apoptotic function, the inventors used the cloned sigma-1 receptor cDNA to resolve this issue. These data confirm that the specific sigma-1 receptor gene product has a potent anti-apoptotic effect (Table 2). Overexpression of the sigma-1 receptor was chosen rather than an antisense approach in the first instance since the sigma-2 receptor (proposed to be pro-apoptotic) has not yet been defined; one candidate for the sigma-2 receptor is a reported splice variant of the sigma-1 receptor. Since the cloned sigma-1 receptor has been confirmed to have an anti-apoptotic function, ligands which bind preferentially to the sigma-1 site and which induce apoptosis can therefore be classed as sigma-1 antagonists since they are inhibiting its anti-apoptotic function. Thus, according to this “working definition” the inventors are now confident to classify rimcazole and IPAG as, at least in part, sigma-1 antagonists in so far as their action on microvascular endothelial cells is concerned.

It has also been deduced that the cloned sigma-1 receptor can act as a general repressor of cell death since for example it can suppress death induction by p53, a molecule which signals to the apoptotic programme possibly through a sigma-independent pathway. Also, sigma-1 suppresses Bax, a molecule close to the final common pathway of death execution. Together, these data tell us that the sigma-1 receptor acts as a general repressor of cell death; this is consistent with the ideas of this and previous inventions: for the sigma-1 receptor to be generally involved in tumorigenesis it must have the property of ability to suppress multiple pathways to death. Thus, one extreme notion would be to view all inducers of cell death as potential “sigma-1 antagonists”, if sigma-1 is a common and important repressor.

Yet we know that the sigma-1 antagonists described in this invention and which induce death in microvascular endothelial cells do not induce death in most cell types, which is an important point of distinction from the majority of inducers of apoptosis; so the agents of the invention are not acting merely to antagonise the action of sigma-1 in its general anti-apoptotic mode.

The synthesis of these data collectively is that the sigma-1 receptor acts both in “private” (proximal or signalling) and “common” (distal) parts of the apoptotic pathway. A private or semi-private pathway is dedicated to early signalling via sigma-1 receptors and its abrogation would provide an initiating stimulus. A common pathway is beyond or close to the point at which divergent signals converge on a final common pathway to death; this is likely to be apoptosis stimulus-independent.

One explanation for cell-selective apoptosis is that it is at least in part due to the specific abrogation of a private arm of the sigma-mediated pathway to survival which exists only in some cell types or in some growth states only.

(+) pentazocine has recently been shown to rescue cortical neurons from death induced by growth and survival factor deprivation; this suggests a general role for the endogenous sigma-1 receptor in apoptotic repression, and an ability for (+)pentazocine to stimulate the general anti-apoptotic function of sigma-1 (Hamabe et al 2000 Cell Mol Neurobiol Vol 20 pp695-702.

Thus, sigma-1 antagonistic ligands are defined by this invention as those agents which (1) specifically bind to sigma-1 sites in classical radioligand binding assays (on isolated cell membranes) and (2) which inhibit survival and/or proliferation in microvascular endothelial cells but not, or to a substantially lesser extent, in normal cells with typical properties of survival and/or proliferation regulation. In this way, they would be distinguished from agents which bind and antagonise the subcellular pool of the sigma-1 receptor which participates in the final common pathway of death induction and which would be anticipated to be non-selective in their ability to induce death.

The agents so defined are not constrained by a necessity to show prevention of death by (+) pentazocine since this agent may be capable of a general role to repress death; also, (+) pentazocine may be of insufficient affinity to rescue antagonists which bind with higher affinity to sigma sites.

It is therefore proposed that all sigma-1 antagonists as defined by this invention will selectively inhibit microvascular endothelial cells and thereby produce an anti-angiogenic effect with little or no toxicity to normal tissues. It is proposed that the term used is “therapeutic sigma-1 antagonistic ligands” to reflect (1) specific binding to sigma sites; (2) selective activity on undesirable cells.

It should nonetheless be appreciated that both antagonistic and agonistic activities are useful within the context of the present invention.

Sigma receptor ligands also include antagonist and agonist antibodies, fragments such as scfv or peptides, e.g. those which are capable of specifically interacting with sigma receptors on endothelial cells and have the property of inhibiting or stimulating endothelial cell proliferation and/or survival.

Antibodies directed to the site of interaction of sigma receptor ligands and sigma receptors can be used as antagonists or agonists for modulating endothelial cell proliferation and/or survival and hence control angiogenesis. Candidate antagonist or agonist antibodies may be characterised and their binding regions determine to provide single chain antibodies and fragments thereof which are responsible for disrupting or promoting the interaction.

Antibodies may be obtained using techniques which are standard in the art and set out below. More generally, methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature 357:80-82, 1992). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies, according to the present invention may be modified in a number of ways. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The mode of determining binding is not a feature of the present invention and those skilled in the art are able to choose a suitable mode according to their preference and general knowledge.

In one preferred embodiment, monoclonal antibodies to sigma 1 receptor and its splice variants can be produced as follows. The endothelial cell sigma 1 receptor ORF or that encoding its splice variant(s) can be cloned downstream of the AcMNPV polyhedrin gene in a suitable plasmid transfer vector. The recombinant transfer plasmid can then be cotransfected with a linearised baculovirus DNA into SF9 insect cells to construct the recombinant baculovirus. Following transfection, a high titre virus stock may be prepared from the appropriate recombinant and a large scale culture prepared and used to produce pure protein or fragments thereof, and used to immunise rats or mice. Protein or peptide and an appropriate adjuvant can be injected into spleens, peritoneal cavities or Peyers patches of the gut and test bleeds carried out following two or three immunisations will determine the titre of antibodies which recognise the sigma 1 receptor its splice variant(s) imobilised on tissue culture wells or columns. Once satisfactory titres are obtained, lymphoid cells are taken and fused with a myeloma cell line to produce hybridomas secreting monoclonal antibodies. Selection of appropriate hybridoma clones depends on the differential binding of culture supernatants to sigma 1 receptor its splice variant(s), and ability to compete with known or new sigma-1 specific ligands in a radioligand binding assay etc. Cells expressing different levels of sigma-1 receptor (selected from a population of cells by ligand binding assays, or engineered using antisense constructs, ribozymes, dominant negative genes or other approaches) are used as a screen to assist with selection of antibodies with desired specificity. Antibodies or peptides with sigma-1 binding selectivity are also selected from phage display libraries of scfv or peptides by “panning” on sigma-1 receptor coated plates. Antibodies with agonistic or antagonistic activity against the receptor are required. These antibodies (or fragments derived from them) can then be employed to assist in predicting the probable effects of ligands or drugs with similar activities on endothelial cell (and tumour cell) behaviour, and potentially minimise problems associated with the sometimes unpredictable behaviour of chiral compounds. Antibodies can also used to design ELISA assays, “capture” assays and to screen for expression of the receptor in human and animal tissues (normal, pathological and malignant). Subcellular localisation is determined using transiently permeabilised cells. “Blocking” antibodies can be used to explore binding of ligands to other (putative) receptors if the sigma 1 receptor is unavailable, and the consequences of this redirected binding. “Agonistic” antibodies may have higher affinity and half-life at the receptor, and can be used as surrogate ligands. Antibodies may be used to target other agents or effectors to the sigma receptor.

### Assay Methods

The present invention also includes a method of identifying a sigma receptor ligand which is an antagonist or agonist capable of modulating endothelial cell proliferation and/or survival, the method comprising:

(a) contacting a test compound with endothelial cells; and,

(b) determining whether the test compound modulates endothelial cell proliferation and/or survival.

In a preferred embodiment, the present invention provides a method of identifying a sigma receptor ligand which is an antagonist or agonist capable of modulating endothelial cell proliferation and/or survival, the method comprising:

(a) contacting a test compound with endothelial cells;

(b) determining whether the test compound modulates endothelial cell proliferation and/or survival; and

(c) where a compound inhibits survival and/or proliferation, determining that the test compound does not, or to a substantially lesser extent, inhibit survival and/or proliferation in “normal” cells, i.e. cells with typical properties of survival and proliferation regulation.

The cells employed in these assays may be large (macro) vascular endothelial cells, microvascular endothelial cells, lymphatic endothelial cells, or populations of cells including one or more of these cell types.

The test compound identified in the assay can then be further tested or developed for use in the modulation of angiogenesis.

The precise format of an assay of the invention may be varied by those of skill in the art using routine skill and knowledge. For example, interaction between substances may be studied in vitro by labelling one with a detectable label and bringing it into contact with the other which has been immobilised on a solid support. An assay according to the present invention preferably takes the form of an in vivo assay. The in vivo assay may be performed in a cell line such as a yeast strain or mammalian cell line in which the relevant polypeptides or peptides are expressed from one or more vectors introduced into the cell.

The amount of test substance or compound which may be added to an assay of the invention will normally be determined by trial and error depending upon the type of compound used. Typically, in vitro binding assays will employ from about 0.01 to 100 nM concentrations of the test compound. Proliferation/survival assays will typically employ 0.1 to 100 μM of the test compound.

Compounds which may be used may be natural or synthetic chemical compounds used in drug screening programmes. Extracts of plants which contain several characterised or uncharacterised components may also be used. Other candidate compounds may be based on modelling the 3-dimensional structure of a polypeptide or peptide fragment and using rational drug design to provide potential inhibitor compounds with particular molecular shape, size and charge characteristics.

Following identification of a substance or agent which modulates or affects endothelial cell proliferation and/or survival, the substance or agent may be investigated further. Furthermore, it may be manufactured and/or used in preparation, i.e. manufacture or formulation, of a composition such as a medicament, pharmaceutical composition or drug. These may be administered to individuals.

### Derivatives

The sigma receptor ligands of the invention can be derivatised in various ways. As used herein “derivatives” of the compounds includes salts, esters and amides, free acids or bases, hydrates, prodrugs or coupling partners.

Salts of the compounds of the invention are preferably physiologically well tolerated and non toxic. Many examples of salts are known to those skilled in the art. Compounds having acidic groups, such as phosphates or sulfates, can form salts with alkaline or alkaline earth metals such as Na, K, Mg and Ca, and with organic amines such as triethylamine and Tris (2-hydroxyethyl)amine. Salts can be formed between compounds with basic groups, e.g. amines, with inorganic acids such as hydrochloric acid, phosphoric acid or sulfuric acid, or organic acids such as acetic acid, citric acid, benzoic acid, fumaric acid, or tartaric acid. Compounds having both acidic and basic groups can form internal salts.

Esters can be formed between hydroxyl or carboxylic acid groups present in the compound and an appropriate carboxylic acid or alcohol reaction partner, using techniques well known in the art.

Derivatives which as prodrugs of the compounds are convertible in vivo or in vitro into one of the compounds. Typically, at least one of the biological activities of compound will be reduced in the prodrug form of the compound, and can be activated by conversion of the prodrug to release the compound or a metabolite of it. One example of the use of prodrug therapy is the use of an antibody specific for a disease marker on a cell coupled to an enzyme capable of converting a prodrug to active drug or toxin.

Other derivatives include coupling partners of the compounds in which the compounds is linked to a coupling partner, e.g. by being chemically coupled to the compound or physically associated with it. Examples of coupling partners include a label or reporter molecule, a supporting substrate, a carrier or transport molecule, an effector, a drug or an inhibitor. Coupling partners can be covalently linked to compounds of the invention via an appropriate functional group on the compound such as a hydroxyl group, a carboxyl group or an amino group.

### Pharmaceutical Compositions

The sigma receptor ligands described herein or their derivatives can be formulated in pharmaceutical compositions, and administered to patients in a variety of forms, in particular to treat conditions which are modulated by the administration of sigma receptor ligand, and more preferably sigma receptor ligand antagonists such as Rimcazole, IPAG, or their salts, derivatives, prodrugs or coupling products as described above.

Pharmaceutical compositions for oral administration may be in tablet, capsule, powder or liquid form. A tablet may include a solid carrier such as gelatin or an adjuvant or an inert diluent. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. Such compositions and preparations generally contain at least 0.1 wt % of the compound.

Parental administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicizing agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parentally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1mM to 50mM. The pH of compositions can otherwise be adjusted by using physiologically acceptable acids or bases.

Isotonicizing agents include sugar alcohols such as glycerol, mannitol or sorbitol; glucose; physiological salts such as sodium, potassium, magnesium or compounds such as NaCl, MgCl₂ or CaCl₂.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a “prophylactically effective amount” or a “therapeutically effective amount” (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980. By way of example, and the compositions are preferably administered to patients in dosages of between about 0.01 and 100mg of active compound per kg of body weight, and more preferably between about 0.5 and 10mg/kg of body weight.

The composition may further comprise one or more other pharmaceutically active agents, in particular further compounds for the treatment of the condition. In the case of treating cancer by inhibiting angiogenesis or endothelial cell proliferation, the medicaments can be administered simultaneously or sequentially with chemotherapy or radiotherapy.

In the normal adult body, most endothelial cells are quiescent, entering mitosis only in response to tissue injury or during menstruation and parturition. In pathological states including psoriasis and diabetic retinopathy, angiogenesis (development of new blood vessels) may occur. In addition, it is now well recognised that for any cancer to grow beyond a few mm in diameter, neoangiogenesis is essential. Tumour cells secrete a variety of angiogenic cytokines which stimulate endothelial cell proliferation. One of the most important is vascular endothelial growth factor (VEGF) which also increases the permeability of newly formed vessels. Thus, angiogenesis is critical for the development of solid cancers, and also provides the conduit through which tumour cells may spread to other parts of the body. Since a small area of capillary can provide nutrients for a relatively large volume of surrounding cancer cells, any inhibition of endothelial cell proliferation will “amplify” the effect on tumour cells.

In addition, there are situations where stimulation of angiogenesis would be of benefit—e.g. to enhance healing of wounds following injury or surgery, in coronary artery disease or tissue ischaemia to stimulate collateral circulation, or to repair damaged blood vessels, e.g. as might result from atherosclerosis. The ischaemia may follow a cerebrovascular or myocardial infarction, an acute thromboembolic episode, chronic vascular ischaemia, angina or peripheral vascular disease. The ligands of the invention may also be employed for stimulation of collateral circulation in the restenosis of vessel grafts, e.g. following bypass surgery. The compositions may be used to control arteriovenous shunts, such as those created surgically in dialysis patients or in congenital arteriovenous malformations. In the former case, sigma ligand antagonists could be employed to inhibit collateral formation, while in the latter it would be beneficial to employ sigma ligand agonist to promote their formation.

Accordingly, the compositions described herein can be used in the treatment of conditions requiring the modulation of angiogenesis or endothelial cell proliferation. In preferred embodiments, the modulation is the inhibition of angiogenesis or endothelial cell proliferation, e.g. in the treatment of conditions such as haemangiomas, diabetic retinopathy, endimetriosis, psoriasis or cutaneous scarring, especially forms of scarring treatable with angiogenesis inhibitors. Sigma receptor ligand antagonists or agonists can also be used to inhibit neovascularisation of tumours, and so can be used to indirectly treat cancer by inhibiting the supply of nutrients to tumour, thereby helping to prevent tumour growth and metastatis.

Such adjuvant therapy using sigma receptor ligand antagonists or agonists may take place as part of the chronic treatment (usually post-operatively) designed to limit or prevent recurrence at the primary site and also metastatic spread, which is often dependent on the generation of new blood vessels. Consequently, if angiogenesis or endothelial cell proliferation is inhibited, metastases can be slowed or prevented.

Rimcazole and its derivatives and other preferred compounds of the invention have the further advantages of low toxicity in vivo even when chronically administered and of good pharmacokinetics such that once or twice daily dosing should be sufficient to obtain therapeutic benefit.

In other embodiments of the invention, sigma receptor ligand antagonists or agonists are used to promote endothelial cell proliferation and/or survival and hence promote angiogenesis. Examples of such conditions include coronary artery disease, the treatment of ulcers (e.g. varicose, gastric or duodenal ulcers), wound healing, ischaemia (e.g. to promote development of a collateral circulation after ischaemic events such as cerebrovascular or myocardial infarction), the repair of damaged or injured tissue, and promoting the integration of tissue grafts.

### Materials and Methods

Sigma Receptor Ligands

The experiments employed rimcazole, IPAG, BD1047, BD1063, haloperidol, cis U50488, (+)-SKF-10047 and (+)-pentazocine. Their properties are discussed further above.

Expression of Sigma 1 Receptor in Human Vascular Endothelial Cells

a) Semi-quantitative RT-PCR to Assay mRNA Levels

The purification of RNA was performed according to standard procedures. In order to eliminate DNA contamination in RNA samples, DNase treatment was routinely employed using 10 units of human placental RNase inhibitor (Amersham Pharmacia Biotech, Bucks, UK) and 10 units of DNase I (Pharmacia) per 10-100 mg of RNA samples. Two mg of RNA sample was annealed to the oligo dT₁₂₋₁₈ primers (Pharmacia) and then underwent the first-strand cDNA synthesis using the Moloney Murine Leukemia Virus reverse transcriptase (Promega, Southampton, UK) according to the protocol of the manufacturer. The final volume of cDNA template was 50 ml. Data regarding gene sequences were obtained from GenBank. Primers for PCR were designed based on strict criteria using the Primer Designer program version 2.0 (S&E Software, PA, USA). Sequences of PCR primer sets (in 5′-3′ direction) were as follows:

5′-GGTACGCAGAGCTTCGTCTT-3′
5′-CCGTACTCCACCATCCATGT-3′
Predicted product size = 431 bp

β-actin was used to check RNA integrity and as an internal control. Typical PCR reaction mixtures contained 5 ml of 10×PCR buffer, 0.25 mM of dNTPs mix, 2.0 units of Red Taq DNA polymerase (Sigma), 200 pM of each upstream and downstream primer, and distilled water to an end-volume of 45 ml. Finally 5 ml of CDNA was added and the reaction mix was overlaid with mineral oil. The PCR reaction was carried out in a TRIO-Thermoblock thermal cycler (Biometra, Gottingen, Germany). Amplification cycles consisted of denaturing the cDNA for 60 s at 94° C., primer annealing for 1.5 min at 55° C. and primer extension during incubation for 1.5 min at 72° C. with the last extension step for 10 min. The optimal condition for each primer pair was achieved by adjusting the annealing/extension temperature and time. PCR products were electrophoresed in 2% agarose gels (NBL Gene Sciences Ltd.) containing 0.5 mg/ml of ethidium bromide. Bands were visualized by examining the gel under UV light and photographed on thermal paper using a Mitsubishi video copy processor.

b) Cloning and Sequencing of Sigma 1 Receptor from Human Vascular Endothelial Cells

RNA was extracted from HUVECs using Promega SV total RNA isolation system. This was then used as a template to prepare cDNA with reverse transcriptase and oligo dT primers using standard methodology. The cDNA was subjected to 28 rounds of PCR using Pfu DNA polymerase using primers designed to amplify the sigma I receptor ORF along with unique NHE1 and EcoR1 sites at the 5′ and 3′ ends of the product. The resulting PCR product was then blunt cloned into the sequencing vector PCRScript Cam (Stratagene). The fidelity of the sigma 1 sequence was confirmed by performing sequencing reactions using standard methodology and comparing it to the published sequence from Genbank.

The sigma receptor ORF has subsequently been cloned into three mammalian expression vectors. Two of the vectors have been designed to allow differential expression of sigma 1 receptor and the third vector will co-express the Renilla GFP protein to enable visualisation of the receptor and its subcellular localisation.

The sigma receptor ORF has additionally been cloned into a baculovirus expression system (Gibco BRL) to enable sufficient recombinant protein to be generated for immunisation of animals for monoclonal antibody production.

Effects of Sigma Ligands on Endothelial Cell Proliferation

HUVECS (human umbilical vein endothelial cells) obtained from TCS Biologicals were grown in TCS Biologicals Large Vessel Endothelial Cell Basal Medium (ZHM-2951) with supplement (ZHS-8945). This contains heparin, hydrocortisone, human EGF, human bFGF (concentrations not disclosed by the Company) with added 2% FCS, pH 7.4.

HMEC-1 (immortalised human microvascular endothelial cells) obtained from Dr Colin Porter, Chester Beatty Laboratories were grown in TCS Biologicals Microvascular Endothelial Cell Basal Medium (ZHM-2946) with supplement (ZHS-8947). This contains heparin, hydrocortisone, human EGF, human bFGF, dibutyryl cyclic AMP (concentrations not disclosed by the Company) with added 5% FCS, pH 7.4.

HDMECS (human dermal microvascular endothelial cells) obtained from TCS Biologicals were grown in TCS Biologicals Microvascular Endothelial Cell Basal Medium (ZHM-2946) with supplement (ZHS-8947). This contains heparin, hydrocortisone, human EGF, human bFGF, dibutyryl cyclic AMP (concentrations not disclosed by the Company) with added 5% FCS, pH 7.4.

The cells were seeded at 2×10⁴ ml in 96 well microtitre plates on day 0, and sigma ligands were added in fresh medium on day 1. Plates were then incubated in a humidified atmosphere of 5% CO₂ in air at 37° C. for 4-7 days and the viability of the cells was determined by the measurement of cell phosphatase activity as below. PCS at the concentrations indicated was present throughout the assay. Each concentration of drug was assayed in triplicate, and the vehicle concentration was kept constant.

Viable cells present at the end of the assay were estimated by determining phosphatase activity as follows: 100Xl of paranitrophenylphosphate (Sigma) at 3 mg/ml in phosphate buffer pH 5.5 containing 0.1% Triton X100 was added to wells and incubated for 2hr at 37° C. The reaction was stopped with 1N NaOH. Released paranitrophenol, indicative of attached cell number, was measured by reading the absorbance at 405 nm in a Titertek multiscan. “Background” counts (which were very low) were subtracted, and the survival in drug treated wells compared with that of control wells. The 100% value is the mean absorbance of control wells treated with vehicle alone; the 0% value is that of wells containing medium only.

In some experiments, agonistic and antagonistic ligands were co-incubated to determine if the former could “rescue” endothelial cells from the anti-proliferative effects of the latter.

Effects of Sigma Ligands on in Vitro Angiogenesis

Human endothelial cells co-cultured with human “feeder” fibroblasts on a fibrin matrix in 24 well plates were incubated with ligands at different concentrations. The ligands were added on days 0, 4, 7 and 9 and the experiment terminated on day 11. On each plate, 2 wells served as untreated controls, and 2 received suramin at 20mM as a standard angiogenesis inhibitor. Each concentration of ligand was assayed in triplicate. On day 11, the wells were washed, and the endothelial cells fixed and stained with anti-CD31 (PECAM-1) antibody and visualised with alkaline phosphatase conjugated second antibody. The cultures were scanned for image analysis and quantitation using a DataCell image analysis system and ImagePro software. Angiogenesis was expressed as the area and/or perimeter of “pseudocapillaries” formed in the gel by the endothelial cells.

Pharmacokinetics of Sigma Ligands in Vivo

Prior to undertaking any in vivo experiments with Rimcazole to study effects on tumour growth and angiogenesis, it was necessary to determine its pharmacokinetics in order that doses in the active range could be administered. Rimcazole (dissolved in water) was administered at 40 mg/kg i.p to Balb/C mice. Heparinised blood samples were taken at 5, 15 and 30 minutes, 1,2,4,6, and 24 hours and plasma separated. Tissue samples were also taken at some time points for analysis. Rimcazole samples were initially analysed by LCMS.

LC Conditions

Column: Supelco 50×4.6 mm ABZ+ with 5 μ packing.

Mobile phase: A=HPLC grade methanol, B=0.1% formic acid in water.

Gradient:

| Time (min) | % A | % B |
|---|---|---|
| 0 | 10 | 90 |
| 0.5 | 10 | 90 |
| 6.5 | 90 | 10 |
| 10.0 | 90 | 10 |

MS Conditions

Full scan mode: mass range analysed 50 to 850 amu.

MSMS mode: filtered ions were 322 (Rimcazole), 338 (Rimcazole+oxygen), and 514 (glucuronide of 338 metabolite).

Effects of Sigma Ligands on Angiogenesis in Vivo

In order to study neoangiogenesis in the absence of tumours, sterile polyether sponges were implanted into animals. This technique, developed in Cambridge, has been widely used to study angiogenesis. We have employed a triple radiotracer technique to study different aspects of the angiogenic process.

a) Studies in Mice

Balb/C mice were implanted bilaterally s.c. with sterile 7 mm diameter polyether sponges on days -5, -12 and -16 days prior to termination (5-6 mice per group). The mice were given Lugol's solution (KI) for 3 days prior to injection of radionuclides to prevent uptake of iodine in the thyroid. 24 hours before termination, the mice were injected i.v with 10Ci [131-I] 5′-iodo-2′-deoxyuridine (IudR). One hour prior to termination the mice were injected with 2 XCi 2-deoxy-D-(1-[H3]glucose (DG) and 2 minutes prior to termination 2XCi [Tc-99m]-exametazine (HMPAO).

The IudR uptake reflects cell proliferation (primarily endothelial cells and also a small proportion of the inflammatory cells which provide the angiogenic growth factors); the DG measures cellular glucose metabolism, and the HMPAO gives a measure of blood flow. All can be taken as separate indices of the degree of vascularisation of the sponge implants. Mice were treated i.p. daily with Rimcazole (30 mg/kg) from the day of sponge implantation, or vehicle alone.

b) Rats

CBH/cbi rats were implanted s.c. with sterile 13 mm diameter sponges as above. They were treated i.p. with Rimcazole (30 mg/kg/day) for 14 days from day 0. Five minutes before termination, they were injected with ¹²⁵I labelled rat albumin (a macromolecule which, in the short term remains intravascular). The amount of radioiodine in the sponges is an index of vascular volume.

Effects of Sigma Ligands on Tumour Growth and Angiogenesis in Vivo

MDA MB 435 human breast carcinoma cells (as a model of hormone-insensitive disease) were grown in tissue culture to 70% confluence, harvested with trypsin:EDTA, washed, resuspended in DMEM and injected bilaterally into the inguinal fat pads of 6 week old female NCr athymic mice (orthotopic site) at 1.5×10⁶ cells in 50 μl. The mice were housed in filter boxes in Maximiser laminar flow cabinets and fed sterilized food and water. All procedures were carried out in class 1 laminar flow hoods using sterile equipment and reagents. On day 7, the tumours were measured with vernier calipers across two perpendicular diameters, and the mice were randomised into experimental groups and treated as follows:

1. Vehicle controls (10% DMSO in sterile double distilled water).

2. Rimcazole 30 mg/kg/day (batch 2, Sigma Aldrich).

3. Rimcazole 15 mg/kg/day (as above).

Rimcazole was dissolved in DMSO, then diluted in water to give a final concentration of DMSO of 10%. The preparations were made up freshly each day, and the animals dosed intraperitoneally for 24 days. Animals were observed daily and weighed regularly to check that there were no adverse effects of treatment. Results were expressed as tumour volume calculated according to the formula V=4/3 π[(d1+d2)4]³ or as tumour volume relative to day 0 values. Differences in growth were compared using the Mann-Whitney U test.

At termination of the experiment the mice were killed and tumours excised and weighed. Blood, liver and tumour samples were snap frozen for analysis of drug levels by LCMS/MS. Tumour samples were also cryopreserved, and sections cut and stained with MECA-32 (Developmental Studies Hybridoma Unit; rat anti mouse endothelium) visualised with HRP conjugated goat-anti rat (Star 72, Serotec) to estimate blood vessel density.

Primary Cell Culture

All primary cells were obtained from Biowhittaker/Clonetics Inc., Walkersville, Md., USA, and grown strictly in accordance with the manufacturer's instructions, using Clonetics specialist media and reagents. (Tissue in all cases was from healthy donors). Independent batches of cells from different donors were studied to ensure reproducibility. Experiments were performed at less than the recommended maximum number of population doublings. Primary cell types included:

Human adult dermal microvascular endothelial cells (catalogue number CC-2543; cells had been characterised by tests for cell type markers: positive for acetylated LDL uptake, positive for factor VIII related antigen, negative for alpha smooth muscle actin).

Human mammary epithelial cells (catalogue number CC-2551; stain positive for cytokeratins 14, 18 and 19).

Human prostate epithelial cells (catalogue number CC-2555; stain positive for cytokeratin 8,13).

Human mammary carcinoma (MDA MB 468) cells were obtained from TCC, Manassas, Va., USA; catalogue number HTB-132; cells were grown in accordance with the manufacturer's instructions.

Cell Viability/Proliferation Assay

This was carried out using an MTS assay (reagents from Promega Corporation, Madison Wis., USA) which is a modification of the MTT assay (as described in Jacobson et al, 1994 EMBO J Vol 13 pp1899-1910). The assay depends on conversion of the MTS tetrazolium compound to a coloured formazan product in metabolically active cells; it is therefore an assay of viable cell number. A decline in values implies cell killing as long as cell disappearance by apoptosis in untreated cell populations is absent or negligible (as with most healthy primary cell populations).

Cells were seeded in the range 1.5×10⁵-1.8×10⁵ cells per ml of culture medium in 96 well microtitre plates and allowed to attach in a humidified atmosphere of 5% CO₂ in air at 37° C.

Drugs were added 18-24 hours later and cell viability/proliferation measured at time intervals up to 48-72 hours post-drug addition when the experiment was terminated. Mean (+/− SE) values at each time point were obtained from wells in triplicate.

Cell viability was measured as follows: 20 ml of MTS solution (Promega) was added to wells and incubated at 37° C. for 3 hours during which time a coloured formazan product is generated in viable cells. (In the MTS assay the formazan product is soluble in tissue culture medium which avoids the solubilisation step required in the MTT assay). Viable cell number was then measured by reading absorbance at 490 nm in a Dynex microtitre plate reader. Cell viability is represented as the ratio of absorbance at time “x” (post drug addition) minus “blank” readings (medium with drug without cells) over absorbance at time zero (prior to drug addition) minus blank readings (medium without drug or cells), expressed as a percentage. 100% reflects viable cell numbers at the start of the experiment; values greater than 100% reflect cell proliferation and values less than 100% reflect cell disappearance (cytotoxicity). These interpretations can be made since values are expressed as a percentage of values at time zero and not relative to control cell populations which will have proliferated in the interim and therefore increased in cell number.

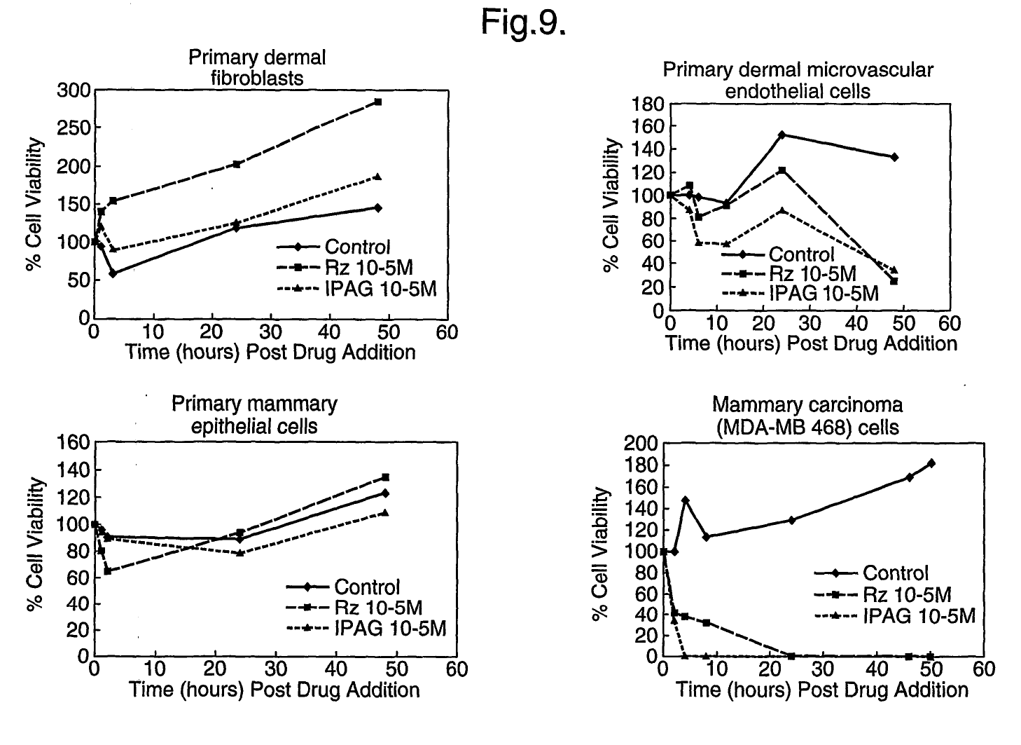

Microvascular Endothelial Cell-selective Killing by Sigma Antagonists (FIG. 9)

A range of primary human cells at low passage and grown in strict accordance with the manufacturer's instructions were compared with human mammary carcinoma (MDA MB 468) cells to determine relative susceptibilities to the sigma antagonistic ligands, rimcazole and IPAG. Cells were seeded in 96-well plates at the same density and allowed to adhere for the same length of time prior to drug addition, to ensure uniformity of conditions. Cell density was checked microscopically at time zero. Culture medium was growth factor and survival factor rich in accordance with the manufacturer's specifications. Cell viability was measured by the MTS assay as described above, at time points up to 48 hours. Sigma ligands were present throughout the duration of the experiment. Representative primary cells (human adult male dermal fibroblasts and human mammary epithelial cells) are depicted in the left hand panels; primary microvascular endothelial cells and mammary tumour cells are depicted in right hand panels. Graphs represent survivability and proliferation rates of the different cell types over a 2 day time course. In all cases, control cell populations (drug vehicle alone) are shown as solid lines; treated cell populations as dotted or dashed lines.

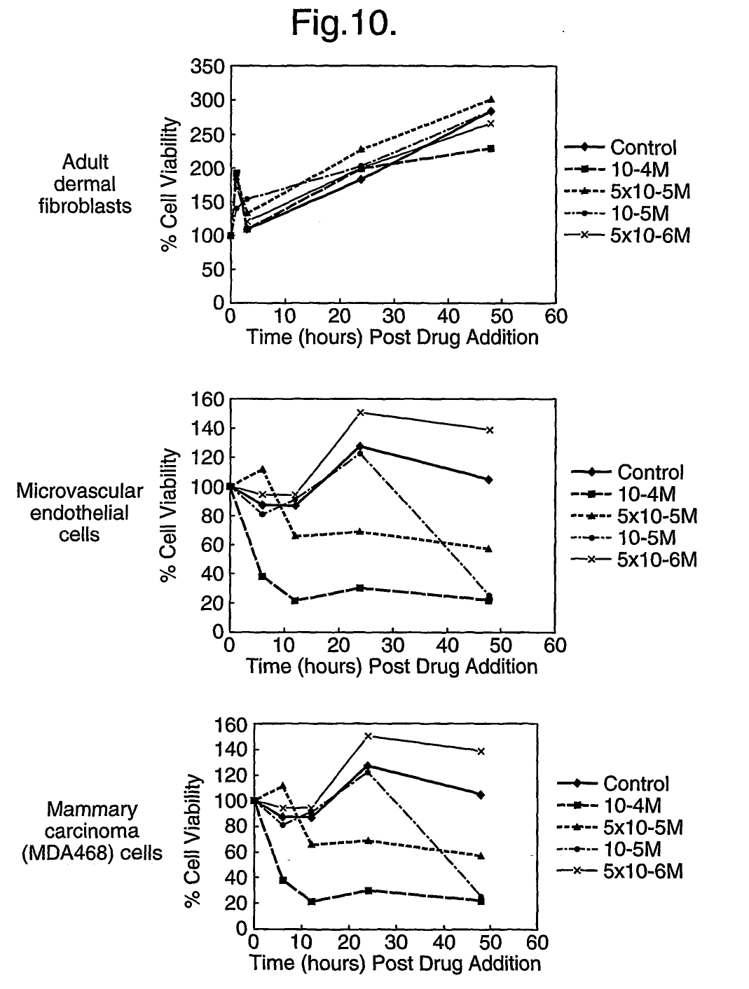

Adult Dermal Fibroblasts Display Robust Resistance Over a Range of Riracazole Concentrations which Induce Dose-dependent Cytotoxicity and Cytostasis in Microvascular Endothelial and Tumour Cells (FIG. 10)

Adult dermal fibroblasts, microvascular endothelial cells and mammary carcinoma cells were treated with a range of concentrations of rimcazole (and IPAG, not shown) from 100 micromolar to the low nanomolar range. Cell viability over a time course was measured in the MTS assay as described above (drug concentrations from 100 micromolar to 5 micromolar are displayed). Control cell populations are shown as solid lines; treated cell populations as dashed or dotted lines.

A Prototypic Sigma-1 Agonist, (+) Pentazocine, Prevents Microvascular Endothelial Cell Death Induction by Rimcazole and IPAG at Equimolar Concentrations (FIG. 11)

Low passage adult dermal microvascular endothelial cells were cultured as described above and exposed to concentrations of rimcazole and IPAG in the presence and absence of the sigma-1 agonist (+)pentazocine. Cell viability was measured over a time course in the MTS assay, as described above. Control cell populations are depicted as solid lines; treated cell populations as dashed/dotted lines.

Sigma-1 Receptor Overexpression Represses p53- and Bax-induced Apoptosis (Table 2)

Human embryonic kidney (HEK) 293 cells were seeded at a density of 10⁵ cells per ml of tissue culture medium and transiently transfected with cytomegalovirus promoter-driven wild-type sigma-1 receptor cDNA (inserted into the plasmid expression vector pcDNA3) using the calcium phosphate method (Ausubel et al 1998 Current Protocols in Molecular Biology; John Wiley & Sons New York). Sigma-1 receptor cDNA (encoding the entire sigma-1 receptor protein product) had been cloned from MCF-7 breast carcinoma cells using a 2 step RT-PCR approach (described above) and its sequence determined to be wild-type.

Sigma-1 receptor cDNA was transfected in the presence and absence of cDNAs encoding two potent inducers of the apoptotic programme, p53 and Bax, also driven by the cytomegalovirus promoter. To exclude non-specific promoter competition effects, the total amount of plasmid DNA was standardised for each transfection using parent vector DNA. Transfection efficiency was estimated using a β-galactosidase-encoding reporter plasmid; in this series of experiments transfection efficiency was approximately 60%. Cells were transfected 2 hours after seeding and tissue culture medium changed 18-24 hours post-transfection. Cells were harvested for analysis at 48 hours after transfection. Estimation of the percentage of apoptotic cells out of the total cell population was performed by FACS analysis of permeabilised propidium iodide stained cells according to standard protocols (as described in WO00/00599). Table 2 represents mean +/− SD apoptotic cell scores as a percentage of the total (transfected and non-transfected) cell population.

### Results

Expression and Cloning of Sigma 1 Receptor from Endothelial Cells

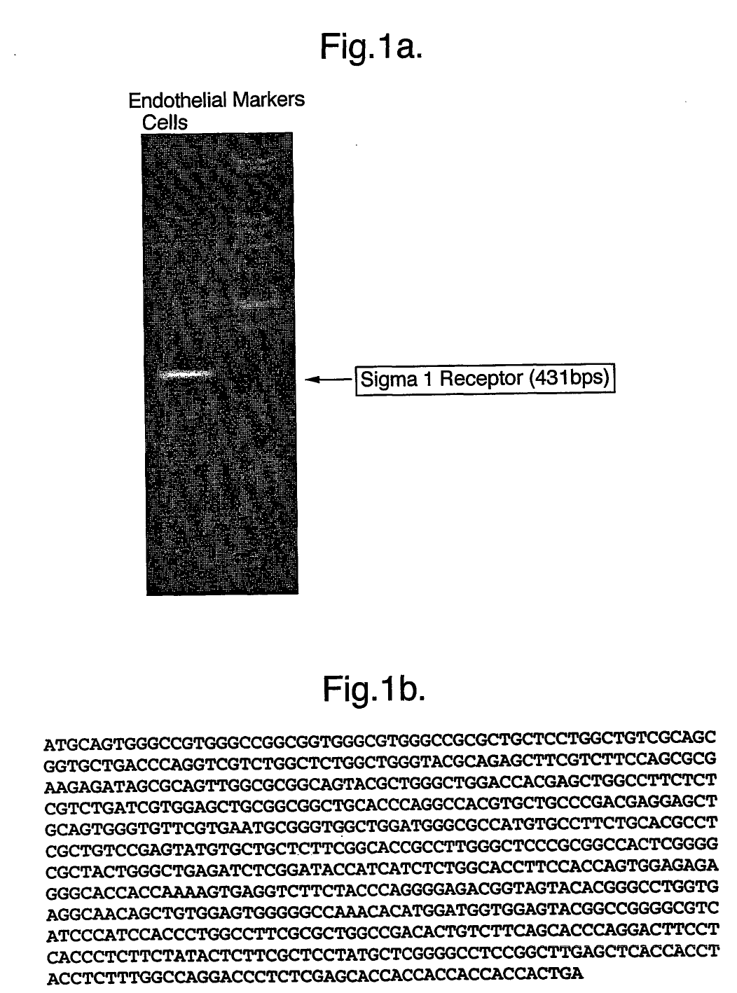

RT-PCR shows strong expression of the mRNA of the sigma 1 receptor in normal human endothelial cells (FIG. 1a).

The gene was cloned from human vascular endothelial cells and the sequence found to match that previously reported and available from GenBank (FIG. 1b).

“Antagonistic” Sigma 1 Ligands Inhibit Endothelial Cell Proliferation

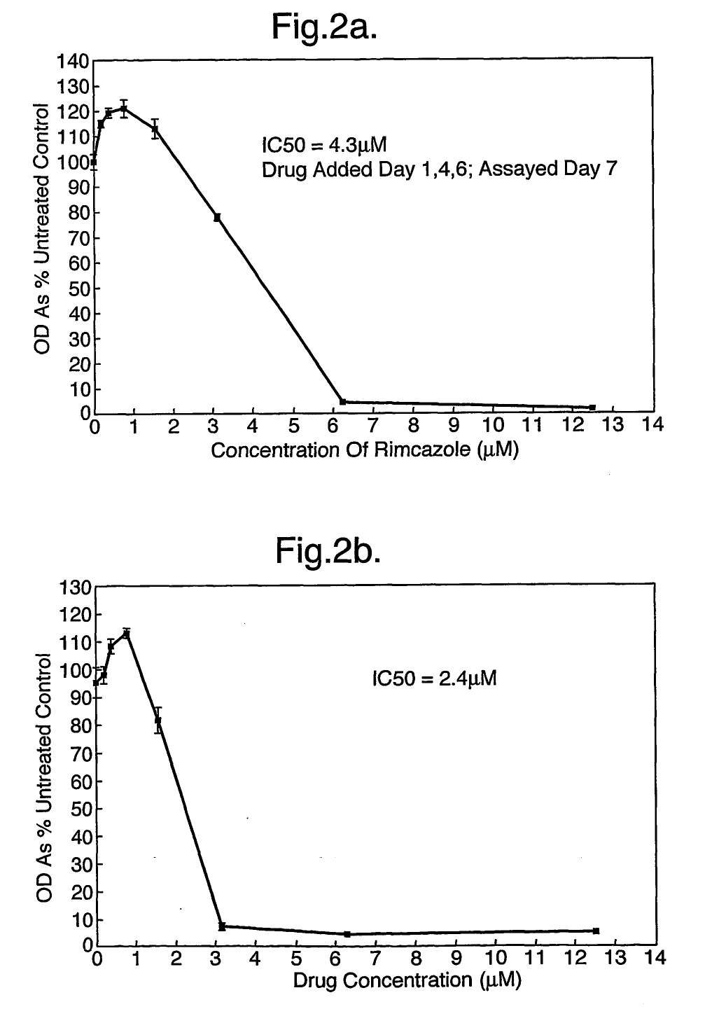

Endothelial cells from two sources proved very sensitive to growth inhibition induced by Rimcazole (FIGS. 2a and b). The IC₅₀ (the dose required to inhibit proliferation to 50% of that of control cells treated with vehicle alone) was less than 5 μM in both cases. IPAG proved as effective as Rimcazole (not shown). HUVEC, HDMEC, and HMEC-1 showed similar responses.

“Agonistic” Sigma Ligands Rescue Endothelial Cells from Inhibitory Effects of Antagonistic Ligands

Ligands reported to have “agonist” activity did not inhibit proliferation of cells, and in fact moderate stimulatory activity was found with (+)-pentazocine and (+)-SKF-10047 (Table 1 below). In other experiments, 4 μM Rimcazole inhibited endothelial cell proliferation down to less than 50% of control cells. However, if this ligand was added together with (+)-pentazocine or (+)-SKF-10047 (4 μM) there was a slight increase in survival, and when a higher dose of the latter ligand (10 μM) was added, the numbers of endothelial cells were the same as in control untreated cultures (FIG. 3).

### Antagonistic Sigma Ligands Inhibit in Vitro Angiogenesis

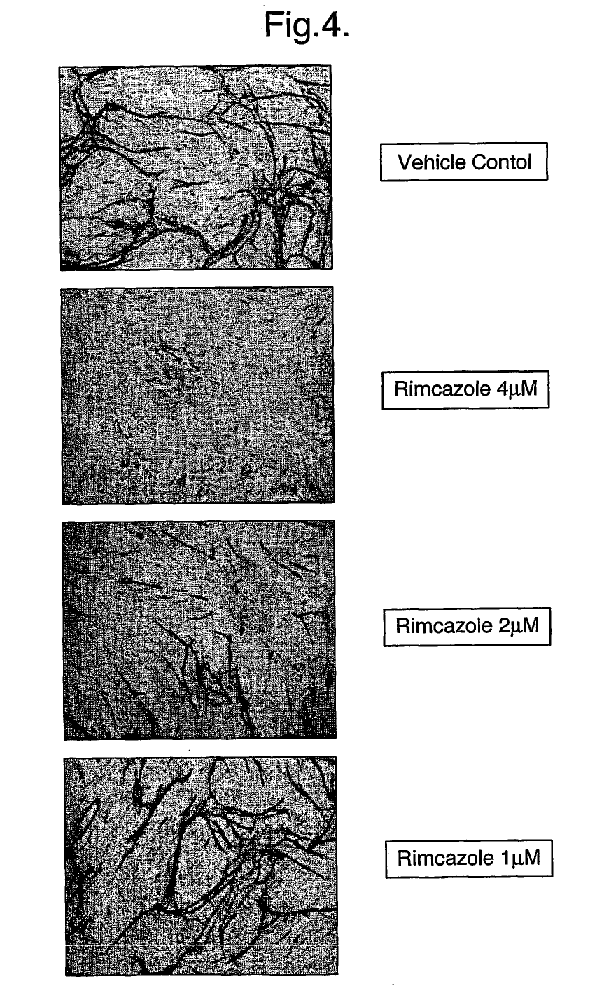

In a more physiologically relevant assay, where endothelial cells are required to undergo differentiation into tubular “pseudocapillary”-like structures in three dimensions, Rimcazole and IPAG were also very effective in inhibiting this function (FIG. 4a and 4b representative images; FIG. 5a and 5b angiogenesis area). Notably, the monolayer of normal fibroblasts which serves as a “feeder” layer for the endothelial cells was apparently undamaged. Pilot studies (FIG. 5c) illustrate that the complete inhibition of angiogenesis induced by 4 μM Rimcazole can be prevented by equimolar concentrations of (+)pentazocine

Rimcazole has Good Pharmacokinetics in Vivo

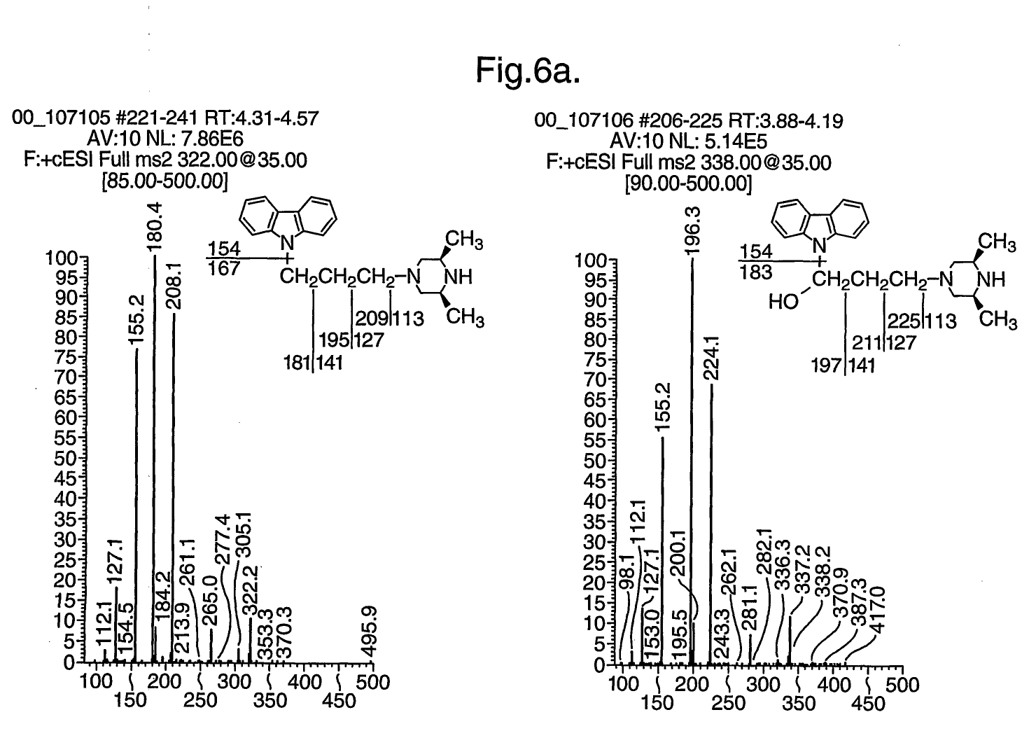

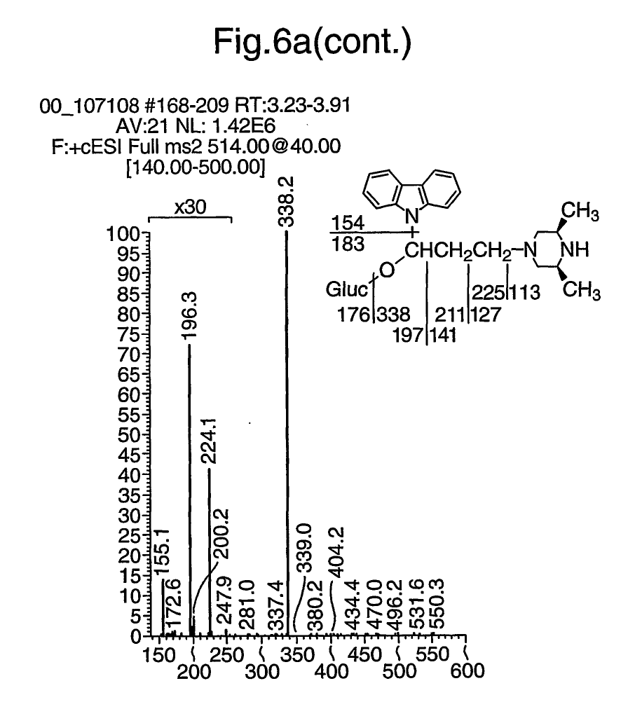

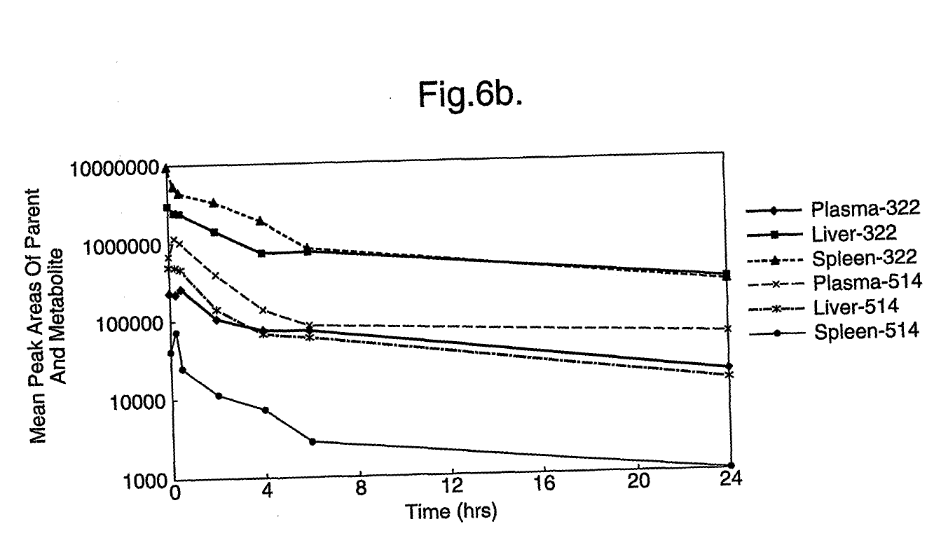

FIG. 6a shows the MSMS spectra for Rimcazole, the hydroxylated metabolite showing the position of hydroxylation (shown in red) and the glucuronide of the hydroxylated metabolite. The structures of all three compounds are shown with lines indicating possible points of fragmentation and the weights of possible fragmentation ions that might be formed. The weights shown are usually one or two amu different to the ions observed in the spectrum because hydrogen rearrangement reactions can occur.

FIG. 6b shows excretion data in plasma, liver and spleen of rimcazole and the glucuronide metabolite (major excretion product). Results show that plasma levels of the glucuronide of the hydroxylated metabolite of rimcazole (labelled plasma—514 on graph) are higher that those of Rimcazole itself (plasma-322). Levels of Rimcazole are higher in spleen and liver than in plasma, but levels of the glucuronide metabolite are much lower than for the parent compound in both tissues. In most cases, compounds can still be detected 24 hr after dosing.

These results indicated that once daily dosing with Rimcazole at 40 mg/kg will give plasma levels above the IC₅₀ for a significant period. Pilot studies, however showed that this dose could not be given repeatedly as the quantity of DMSO in the vehicle was an irritant in the peritoneal cavity. In vivo therapy experiments were therefore carried out using doses of 30 mg/kg or lower. Recently, we have also found that IPAG has similarly favourable PK (not shown) and hence could be used in vivo.

Rimcazole Inhibits Neoangiogenesis in Vivo

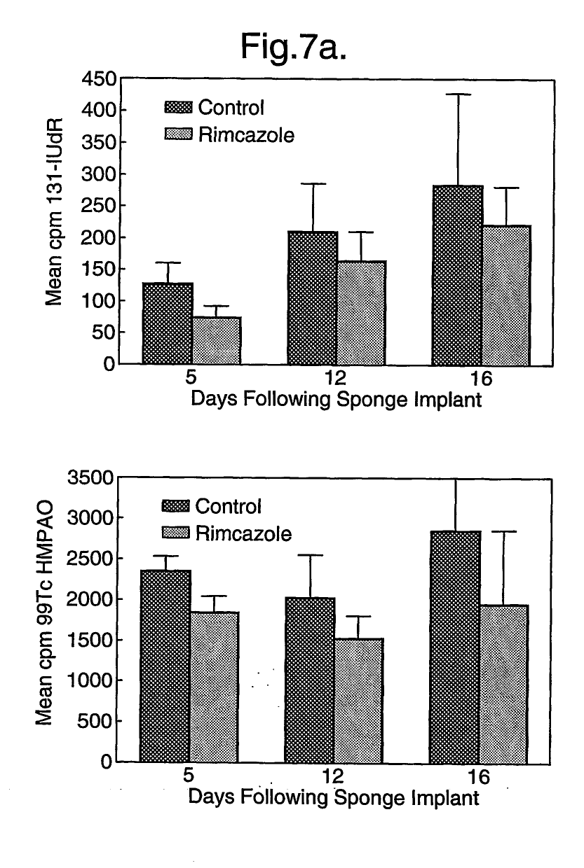

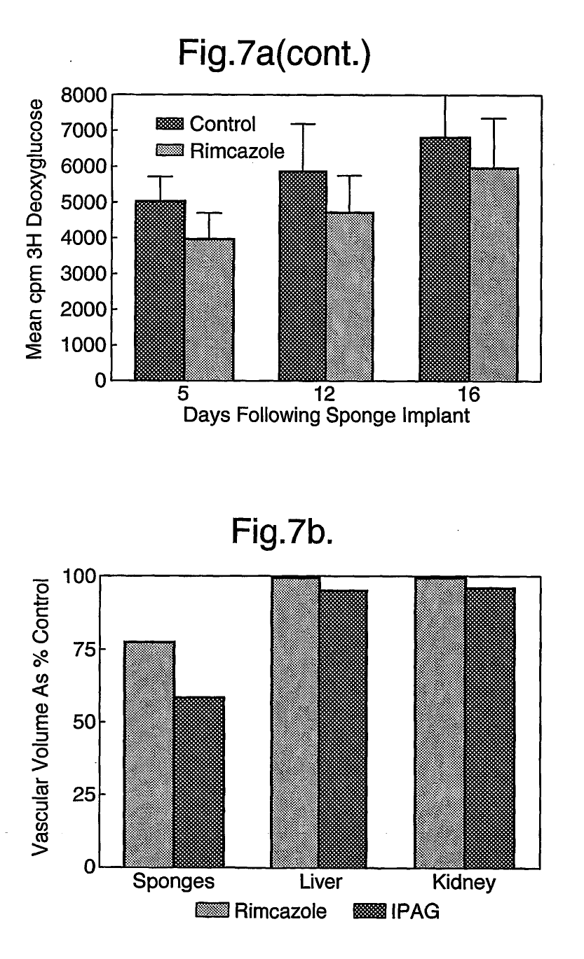

FIG. 7a shows that in the mouse “sponge” angiogenesis model, all three parameters assayed (cell proliferation, cellular metabolism and blood flow) consistently showed a reduction in mice treated with Rimcazole at all time points assayed.

FIG. 7b shows that in the rat “sponge” angiogenesis model, neoangiogenesis (as assayed by localisation of radiolabelled albumin which measures vascular volume) was reduced by both Rimcazole and IPAG administered at 30 mg/kg/day for 14 days after implantation. The vascular volume of normal organs (liver and kidney) was unaffected, showing that normal blood vessels were not compromised.

Rimcazole Inhibits the Growth of Established Human Breast Carcinoma Xenografts in a Dose Dependent Manner

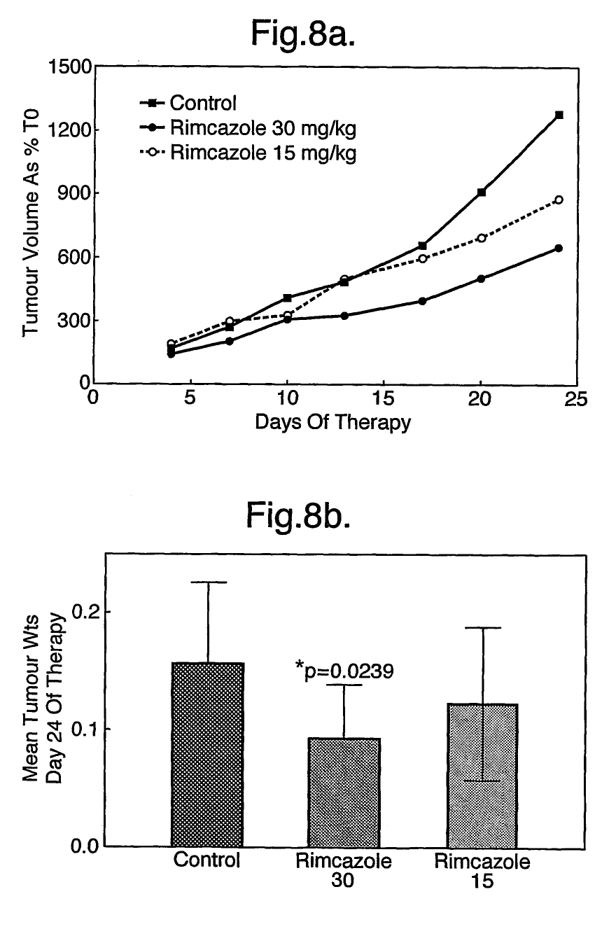

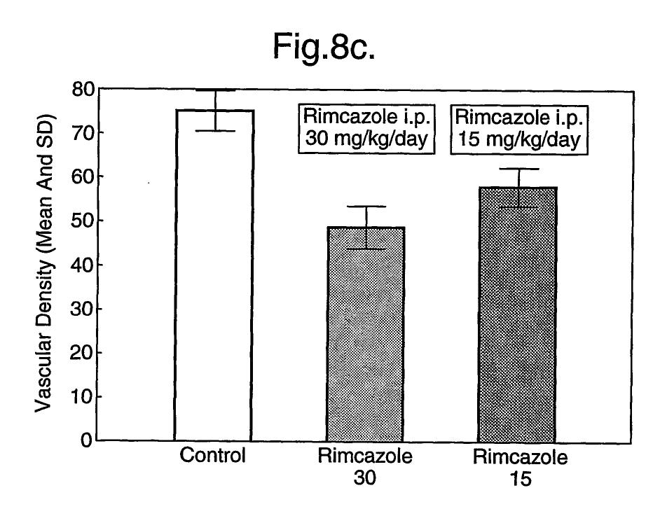

FIG. 8a shows the growth of MDA MB 435 breast carcinoma xenografts in mice treated with vehicle, or Rimcazole at two dose levels. Therapy was commenced once tumours were established and the results are expressed as the percentage increase in volume relative to tumour volumes in each group at the start of treatment. Daily doses of 30 mg/kg/day and 15 mg/kg/day for 24 days resulted in significant tumour growth retardation (p=0.0436 and 0.0228 respectively, Mann-Whitney U test).

FIG. 8b shows that the weights of excised tumours were also significantly less in the higher dose group (p=0.0239) but did not achieve statistical significance at the 15 mg/kg Rimcazole dose. The mice remained healthy throughout therapy and gained weight at the same rate as controls (data not shown).

FIG. 8c shows that in the excised tumours, the vascular density was significantly inhibited by both doses of rimcazole, consistent with an anti-angiogenic component of growth inhibition.

Microvascular Endothelial Cell-selective Killing by Sigma Antagonists (FIG. 9)

Mammary carcinoma (MDA MB 468) cells were killed decisively by 10 micromolar concentrations of rimcazole and IPAG; no surviving cells remained after 24 hours (bottom right hand panel, FIG. 9). These tumour cells were at “low passage” (approximately passage 350—“low” for a tumour cell line); low passage tumour cells appeared more susceptible than later passage tumour cells. The inventors believe that when tumour cells spend an extended time in culture in a survival factor-rich environment they may lose selective pressure to maintain high levels of sigma receptor expression; enhanced susceptibility of low passage tumour cells bodes well for susceptibility of tumours in the authentic clinical situation. In contrast, adult male dermal fibroblasts and adult mammary epithelial cells not only survived but continued to proliferate vigorously in the presence of 10 micromolar concentrations of rimcazole and IPAG.

These data confirm that microvascular endothelial cells are unusual, compared to other primary cells at a similar stage of maturity and also in a guaranteed primary state, in being unduly susceptible to sigma antagonistic ligands. A decline in MTS scores (compared to MTS values in cells prior to drug addition, at time zero) confirms cell disappearance and hence cytotoxicity.

Adult Dermal Fibroblasts Display Robust Resistance Over a Range of Rimcazole Concentrations which Induce Dose-dependent Cytotoxicity and Cytostasis in Microvascular Endothelial and Tumour Cells (FIG. 10)

Measurement of viable cell number over a time course, as in the MTS assay, revealed that adult dermal fibroblasts were remarkably resistant to rimcazole even up to high (100 micromolar) concentrations; not only was viability maintained, cell proliferation rates appeared indistinguishable from control (untreated) cells (FIG. 10, top panel). Fibroblasts tolerated rimcazole over a wide range of concentrations, from 100 micromolar to 1 nanomolar; thus, paradoxical effects at inappropriately high doses could be excluded. Cells were slightly less tolerant of high concentrations of IPAG but were still remarkably resistant over a wide range of concentrations. The extent of normal cell sparing shows that the treatment has low toxicity and therefore has wide applicability in non-life-threatening diseases.

FIG. 10 also illustrates dose-dependent killing and cytostasis induced by rimcazole and IPAG in both microvascular endothelial cells and tumour cells. In mammary carcinoma cells, cytostasis of cells was seen with sublethal concentrations of drug (as in FIG. 10 bottom panel, rimcazole, 5 micromolar). Similarly, concentrations of rimcazole which did not kill the entire population of microvascular endothelial cells, induced stasis in the viable fraction of cells (FIG. 10, middle panel, 12-24 hours, whilst control cells are actively dividing). Thus, sigma antagonists will prevent the formation of new vascular networks since this requires endothelial cell proliferation.

Beyond 24 hours, control microvascular cell populations were no longer dividing; despite this, rimcazole at 10 micromolar concentrations induced an approximately 80% reduction in viability between 24 and 48 hours. Thus, sigma antagonists are effective even on non-dividing microvascular endothelial cells and thus will be effective in causing regression of already established neovascular networks. Thus, sigma antagonists are indicated for established angiogenesis as for example in advanced diabetic retinopathy, in addition to early stage disease where prevention of new networks will arrest or slow the course of the disease.

It should be noted that in this series of experiments cells were seeded at a higher density (5-10 fold approximately) than in other experiments described herein. In the experiments above, a higher cell density was required in order to obtain recordable values in baseline cells with which other values were compared (this was required in order to assess changes in cell number over time, i.e. proliferation rates; also, whether cells were declining in number with respect to baseline). As a result, microvascular endothelial cells in these experiments are apparently less sensitive to sigma antagonists than in other experiments. It is important to note that this is not due to intrinsic variation in the sensitivity of microvascular cells, but instead is due to the modulatory effect of diffusible and non-diffusible extracellular survival factors which are present at higher levels in high density cell cultures. At a given cell density, under identical culture conditions, and at low passage, microvascular endothelial cells are very consistent in their degree of susceptibility to sigma ligands. This knowledge will facilitate the design of screens to identify new agents with higher potency.

A Prototypic Sigma-1 Agonist, (+) Pentazocine, Prevents Microvascular Endothelial Cell Death Induction by Rimcazole and IPAG at Equimolar Concentrations (FIG. 11)

Treatment of cells with 10 micromolar concentrations of IPAG and rimcazole induced a 60-80% reduction in viable cell numbers compared to baseline values; this confirms a cytotoxic effect (cell disappearance). This cytotoxic effect was completely prevented by co-administration of the sigma-1 agonist (+) pentazocine, also at 10 micromolar concentrations; furthermore, cells continued to proliferate. A lesser attentuation of death at higher concentrations of sigma antagonists was observed. Interestingly, co-administration of (+) pentazocine with both rimcazole and IPAG increased cell proliferation rates above control values. This indicates a potential for promotion of angiogenesis through enhanced endothelial cell proliferation when sigma-1 agonists are combined with rimcazole and IPAG (see also Table 1).

Sigma-1 Receptor Overexpression Represses p53- and Bax-induced Apoptosis (Table 2)

HEK 293 cells were chosen as a model system since they can be transiently transfected with high efficiency; overexpression of a gene product can be induced for a sufficient length of time to perform functional studies. Endothelial cells can not be transfected with high efficiency. It is the aim of the inventors to achieve high level expression of the sigma-1 receptor in microvascular endothelial cells using adenoviral gene transfer but regulatory authority approval for such studies must be obtained; this was out with the time scale of the exemplification period. HEK 293 cells were therefore chosen as a model system. This was consistent with the ideas of the invention since it is the thesis of this and previous inventions (WO96/06863 and WO00/00599) that all cell types will be responsive to provision of an excessive anti-apoptotic drive mediated through opioid-like and in particular the sigma-1 receptor. (But only restricted cell types will be sensitive to abrogation of survival mediated through this pathway, due to undue reliance on sigma-1-mediated survival). Furthermore, a general role in tumorigenesis, as has been predicted and exemplified (WO96/06863 and WO00/00599) would require this pathway to be generally effective against a range of inducers of apoptosis.

For a molecule to be effective as an inhibitor of diverse stimuli to apoptosis it must act at least in part close to the final common pathway of death execution (that is, beyond the point at which diverse signalling pathways converge on a common apoptotic pathway). A pro-apoptotic member of the Bcl-2 family, Bax, acts close to the final common pathway of apoptotic execution; for example, Bax has been shown to induce the release of cytochrome C from mitochondria (Jurgensmeier et al 1998 PNAS Vol 95 pp4997-5002). Thus, if the sigma-1 receptor is a general repressor of cell death it should suppress the pro-apoptotic function of a protein such as Bax. It should also repress the apoptotic function of molecules which act further upstream as decision-makers, such as p53.

The inventors have determined that overexpression of the wild-type full-length sigma-1 receptor protein potently represses the apoptotic function of p53 and Bax (Table 2), in a dose dependent manner. A small induction of apoptosis in the presence of sigma-1 alone (to 6-7%) is due to a non-specific effect of the calcium phosphate-mediated transfection procedure since it was equally apparent in parent vector alone samples; also, it was no greater with greater amounts of sigma-1 receptor cDNA. The overexpression of sigma 1 on its own therefore had no significant effect on cell viability. P53 and Bax in contrast induced significant amounts of apoptosis (approximately one-third and two-thirds of the transfected cell population which was approximately 60% of the total cell population). When the sigma-1 receptor was co-transfected with p53 or Bax cDNAs, there was a significant reduction in apoptosis by more than 80% when the greater amount of sigma-1 was co-transfected with p53 and by approximately 70% when co-transfected with Bax. Since transient overexpression of the sigma-1 receptor confers an anti-apoptotic function this indicates that natural ligand(s) for the sigma-1 receptor, which have thus far not been identified, are present in non-limiting amounts. These data therefore confirm that the sigma-1 receptor is a potent general repressor of cell death which explains the undue reliance of microvascular endothelial cells, required to survive in adverse circumstances, on such a pathway.

TABLE 1

| Ligand | Antagonists: IC50 | Agonists: stimulation |
|---|---|---|
| Rimcazole | 5 μM | |
| IPAG | 11 μM | |
| BD1047 | 74 μM | |
| BD1063 | 71 μM | |
| Haloperidol | 10 μM | |
| Cis U50488* | 97 μM | |
| (+)-SKF-10047 | | 110% at 100 μM |
| (+)pentazocine | | 120% at 4 μM |

IC50: concentration of drug required to inhibit survival and/or proliferation by 50%.
Stimulation: increase in cell number relative to controls on day 4.
*Cis-U50488 - the functional properties of cis-U50488 are discussed above.
Note:
the lesser potency of BD-1047 and BD-1063 is likely to be due to their ability to bind both sigma-1 and sigma-2 sites with high affinity although they have a preference for sigma-1 sites (see above). It has been reported that specific sigma-2 agonists are pro-apoptotic (Vilner and Bowen 2000 JPET Vol 292 pp900-911); thus, antagonistic action at the sigma-2 site will offset antagonistic action at the sigma-1 site.

TABLE 2
Sigma-1 Receptor Overexpression Represses p53- and Bax
Induced Apoptosis
% Apoptosis ± SD

| DNA | | | |
|---|---|---|---|
| Transfected | | p53 5 μg | Bax 5 μg |
| | | 22.06 ± 0.22 | 42.68 ± 0.4 |
| Sigma-1 10 μg | 6.79 ± 0.26 | 11.04 ± 1.08 | 18.65 ± 1.42 |
| Sigma-1 20 μg | 6.69 ± 0.13 | 3.96 ± 0.25 | 13.94 ± 0.01 |
| Control | 2.73 ± 0.11 | | |

The references mentioned herein are all expressly incorporated by reference.

1. Use of a sigma receptor ligand for the preparation of a medicament for modulating endothelial cell proliferation and/or survival.

2. The use of claim 1, wherein ligand is capable of binding a sigma-1 receptor.

3. The use of claim 1 or claim 2, wherein the endothelial cells comprise microvascular endothelial cells, macrovascular endothelial cells and/or lymphendothelial cells.

4. The use of any one of claims 1 to 3, wherein modulating endothelial cell proliferation and/or survival is used to control angiogenesis.

5. The use of any one of claims 1 to 4, wherein the sigma receptor ligand is a sigma receptor antagonist which inhibits endothelial cell proliferation and/or survival.

6. The use of claim 5, wherein the sigma receptor ligand antagonist is rimcazole (cis-9-[3,5-dimethyl-1-piperazinyl) propyl]carbazole dihydrochloride), rimcazole hydrochloride or IPAG (1-(4-iodophenyl)-3-(2-adamantyl) guanidine, or a derivative, prodrug or pharmaceutically active salt of any one of said compounds.

7. The use of claim 5 or claims 6, wherein the medicament is employed for the treatment of cancer.

8. The use of claim 7, wherein the medicament inhibits neovascularisation of tumours, thereby inhibiting tumour growth and metastasis.

9. The use of claim 5 or claim 6, wherein the medicament is employed for the treatment of haemangiomas, diabetic retinopathy, endimetriosis, psoriasis, cutaneous scarring or venous shunts.

10. The use of any one of claims 1 to 4, wherein the sigma receptor ligand is a sigma receptor agonist which promotes endothelial cell proliferation and/or survival.

11. The use of claim 10, wherein the sigma receptor ligand agonist is (+)-N-allyl normetazocine or (+)pentazocine.

12. The use of claim 10 or claim 11, wherein the medicament is employed for the treatment of coronary artery disease, ulcers, wound healing, ischaemia, to repair of damaged or injured tissue or to promote the integration of tissue grafts.

13. The use of claim 12, wherein the ulcers are varicose, gastric or duodenal ulcers.

14. The use of claim 12, wherein the ischaemia follows a cerebrovascular or myocardial infarction, an acute thromboembolic episode, chronic vascular ischaemia, angina or peripheral vascular disease.

15. The use of claim 12, wherein the damaged tissue comprises blood vessels.

16. The use of claim 15, wherein the damage results from atherosclerosis, damage from emboli, venous shunts or restenosis.

17. A method of identifying a sigma receptor ligand which is an antagonist or agonist capable of modulating endothelial cell proliferation and/or survival, the method comprising:
(a) contacting a test compound with endothelial cells;
(b) determining whether the test compound modulates endothelial cell proliferation and/or survival; and
(c) where a compound inhibits survival and/or proliferation, determining that the test compound does not, or to a substantially lesser extent, inhibit survival and/or proliferation in normal cells.
